(19) United States
(12) Patent Application Publication
(10) Pub. No.: US 2026/0102633 A1
(43) Pub. Date: Apr. 16, 2026

(54) ROTATABLE PATIENT TABLE FOR RT APPLICATIONS

(71) Applicant: Siemens Healthineers AG, Forchheim (DE)

(72) Inventor: Wolfgang NEUBER, Eschenbach i. d. Opf. (DE)

(73) Assignee: Siemens Healthineers AG, Forchheim (DE)

(21) Appl. No.: 19/357,797

(22) Filed: Oct. 14, 2025

(30) Foreign Application Priority Data
Oct. 15, 2024 (DE) 10 2024 209979.1

(51) Int. Cl. A61N 5/10

(52) U.S. Cl. CPC A61N 5/1069

(57) ABSTRACT

A patient table has a lower structure and an upper structure, which are connected via a lifting mechanism. A table top is on the upper structure, on which an adult person can be supported. At one of its ends the lower structure is supported on a foundation via a pivot bearing and can consequently rotate about a vertical axis of rotation. The lifting mechanism is arranged between the two ends of the lower structure and below the upper structure. Based on the axis of rotation, a gear casing is arranged radially inside the pivot bearing and axially in the region of the pivot bearing. An arm acts on the lower structure via a coupling element, via which the torque exerted on the arm is transferred to the lower structure while a radial movement of the arm relative to the lower structure is enabled.

### CROSS-REFERENCE TO RELATED APPLICATION(S)

The present application claims priority under 35 U.S.C. § 119 to German Patent Application No. 10 2024 209 979.1, filed Oct. 15, 2024, the entire contents of which are incorporated herein by reference.

### RELATED ART

A patient table is generally known.

In the case of a patient table for radiotherapy applications (what is known as an RT couch), firstly it is necessary to be able to position a table top and a beam generator exactly relative to each other and secondly, as freely as possible. In some cases, the foundation is immovable relative to the space in which the beam generator is arranged. In other cases it is a rotary plate. In a case, a pivot bearing is located as far as possible at the edge of a rotary plate.

Owing to the arrangement of the pivot bearing at the end of a lower structure, it is possible for a high tilting moment to act on the pivot bearing. It must be possible for this tilting moment to be received and absorbed by the pivot bearing. Appropriate pivot bearings are known as such. In practice, however, there is the additional problem that the possible installation space—in particular the installation height—has to be kept very small/low in order to attain appropriate freedom of movement for a rotating RT gantry. Furthermore, it is necessary for the lower structure (and all components arranged on the lower structure) to be able to rotate very exactly about the axis of rotation. Furthermore, in addition to the mechanics in the region of the pivot bearing, there must also be a cable feedthrough.

What is known as a compact gear with integrated bearing is used in the prior art for mechanical axes with similar requirements (high moment owing to an asymmetric load application, high precision and strictly limited installation space). One example of such a gear is what is known as a cycloidal gear. In this case, there are complete gear units which, as standardized components, can be adapted to the machine interfaces. Both the bearing and the gear are present in this component. The size of this component is determined by the tilting moment in this case, which has to be received and absorbed by the component.

In the case of a patient table, a weight of the patient of 300 kg is customarily anticipated, sometimes even slightly more. The tilting moment is very large owing to the, in respect of the axis of rotation, eccentric bearing of the patient on the patient table and the relatively high inherent weight of the patient table. Corresponding herewith, the component mentioned above must also be of sufficiently large dimensions. However, this opposes the free movement of the beam generator.

### SUMMARY

According to one or more example embodiments, a patient table in which the size of table, which comprises both the pivot bearing and the gear, is reduced.

The object is achieved by a patient table with the features of claim 1. Advantageous embodiments of the patient table are the subject matter of dependent claims 2 to 12.

### BRIEF DESCRIPTION OF THE DRAWINGS

Properties, features and advantages of example embodiments and the manner in which they are achieved will become clearer and more comprehensible in conjunction with the following description which are explained in more detail in connection with the drawings. In the drawings in a schematic representation:

FIG. 1 shows a patient table according to one or more example embodiments,

FIG. 2 shows a perspective representation of part of a lower structure of a patient table and of part of a foundation according to one or more example embodiments,

FIG. 3 shows a sectional view through a lower structure and a foundation and components, arranged there, of the patient table according to one or more example embodiments,

FIG. 4 shows a view of an arm and a coupling element from the side according to one or more example embodiments,

FIG. 5 shows a sectional view along a line V-V in FIG. 4 according to one or more example embodiments,

FIG. 6 shows a plan view of a lower structure according to one or more example embodiments,

FIG. 7 shows a plan view of a lower structure according to one or more example embodiments,

FIGS. 8 and 9 shows plan views analogous to FIG. 7 in other rotational positions of the lower structure according to one or more example embodiments, and

FIG. 10 shows a cable drum and sections of a cable according to one or more example embodiments.

### DETAILED DESCRIPTION

Inventively, a patient table of the type mentioned in the introduction is embodied in that
the gear is a structural element different from the pivot bearing,
based on the axis of rotation, the gear casing is arranged radially inside the pivot bearing and axially in the region of the pivot bearing and
the arm acts on the lower structure via a coupling element, via which the torque exerted on the arm is transferred to the lower structure, although a radial movement of the arm relative to the lower structure is still enabled.

One or more example embodiments is therefore based, on the one hand, on the idea of separating the two required functionalities from one another—i.e. firstly, the rotatable bearing and secondly, the application of the torque necessary for rotation with a simultaneously precise setting of the angle of rotation. Since the torque is relatively low, the gear can be small in design. In particular, owing to the separation, it is possible to arrange the gear casing radially inside the pivot bearing and axially in the region of the pivot bearing. The type of gear can be determined as needed. It can be, for example, a cycloidal gear, what is known as a strain wave gear, a planetary gear or another type of axial gear.

However, due to the implementation in two structural elements that are different from each other, a very precise alignment of the gear casing relative to the pivot bearing would be necessary with a rigid connection of the arm to the lower structure. In particular, the axis of rotation of the driven shaft would have to be located exactly coaxially with the axis of rotation of the pivot bearing. It is difficult to achieve a very precise alignment of this kind in practice. However, a very precise alignment is no longer necessary due to the coupling element, which acts as a torque support and permits the radial movement of the arm relative to the lower structure. The small offset of the axis of rotation of the driven shaft relative to the axis of rotation of the pivot bearing—in practice it is usually 1 mm or less—can readily be compensated by an appropriate (small) radial movement of the arm. As a result of the present invention it will therefore be unproblematic if there is a small offset. A constant rotational resistance of the gear still results over the entire range of rotation. At the same time the precision requirements placed on the assembly interfaces can be kept low.

When the lower structure is rotated, it is not just the lower structure itself which rotates, rather the lifting mechanism and the upper structure also rotate with the lower structure about the vertical axis of rotation relative to the foundation. Furthermore, the table top can be movable (at least) in its longitudinal direction. In this case—depending on the direction of motion—the tilting moment which acts on the pivot bearing can be significantly increased once again, or else be reduced. The pivot bearing must of course be designed for the worst case scenario.

Various solutions are possible for the embodiment of the coupling element. A simple solution consists in embodying the coupling element as a slot with a radially-running longitudinal axis. In this case, a pin of the arm is led into the slot. Similarly, it is conversely possible to embody the coupling element as a pin. In this case, the pin is led into a slot, introduced into the arm, with a radially-running longitudinal axis. In both cases the pin extends in the axial direction, based on the axis of rotation. Another solution consists in embodying the coupling element as a lever, which extends substantially tangentially to the axis of rotation, with two ends. In this case, one each of the two ends of the lever is articulated to the lower structure and the arm.

Other solutions are also possible. For example, the coupling element can also be embodied as an elastic tensile/pressure element, for example as a sheet metal strip. A fastening free from play of a sheet metal strip of this kind to the arm and to the lower structure results in a simple transmission of the torque which is free from play and at the same time rigid.

The pivot bearing has an internal diameter. Similarly, the gear casing has a casing diameter. However, the casing diameter of the gear casing is smaller than the internal diameter of the pivot bearing. An annular gap is consequently formed between the pivot bearing and the gear casing. In a preferred embodiment, a feedthrough is arranged in the annular gap, via which feedthrough a cable is guided from the foundation into the lower structure. The drive is supplied with electrical energy via the cable. Data (for example, actual positions or actual current values) can optionally also be transmitted from the drive back to a control facility (also referred to as a controller).

The lower structure can customarily rotate between two end positions via the drive. Preferably, when the lower structure is in a middle rotational position between the two end positions, the gear casing is arranged between the other end of the lower structure and the feedthrough. This embodiment has proven to be particularly reliable for the cable in continuous operation.

Preferably, the drive is also arranged between the other end of the lower structure and the pivot bearing. This is advantageous for constructional reasons and, in particular, in relation to the resulting installation height.

As a rule, the pivot bearing has an external diameter. In this case, the spacing of the drive from the axis of rotation is preferably greater than the external diameter.

In one possible embodiment, the cable is embodied as a cable carrier which, depending on the direction in which the lower structure is rotated via the drive, is placed in the annular gap or removed from the annular gap. This embodiment operates reliably and is particularly compact. In particular, no additional installation space is required above the gear and the pivot bearing for receiving the cable.

Preferably, the cable is also arranged in a protective sheath. In this case, the protective sheath, together with the cable, is placed in the annular gap or removed from the annular gap. The cable therefore always remains in the protective sheath.

In a further embodiment, a first end section of the cable is securely arranged on the lower structure and a second end section of the cable is also arranged in the region of the feedthrough. In this case, a cable drum is supported on the lower structure, so the cable drum can move translationally and can rotate about a drum axis. A drum section of the cable, which is located between the first and the second end sections, is fixed inside the cable drum. When the cable drum rotates about the drum axis, depending on the direction of rotation of the cable drum, the cable drum either unwinds or winds the cable both in the direction of the first as well as the second end section. This embodiment has the advantage that bending radii of the cable, which dynamically change during winding and unwinding, are limited in the direction of small bending radii by the cable drum. Very small bending radii can occur in the drum section itself but these bending radii are static. Bending only occurs once here therefore; bending does not occur again with each winding and unwinding. The drum axis can be oriented as needed. It is often oriented either vertically or horizontally, with the former being preferred. The embodiment with the cable drum can be implemented with a very compact design. Furthermore, the cable drum can be pre-assembled as a stand-alone unit. The cable is only stressed when tensioned. A torsion does not occur.

A first contact region of the cable drum is defined in that at this point of the cable drum, in the direction of the first end section, a first section of the cable wound onto the cable drum merges into a section of the cable which extends in the direction of the first end section. Analogously, a second contact region of the cable drum is defined in that at this point, in the direction of the second end section, a second section of the cable wound onto the cable drum merges into a section of the cable which extends in the direction of the second end section. Preferably, the first and the second contact regions of the cable drum are located approximately diametrically opposed in respect of the drum axis. The cable drum can consequently output the cable in the direction of both the first and the second end sections with an at least substantially “correct” alignment.

Preferably, the cable drum has a first storage area and a second storage area. The first storage area serves to wind the section of the cable which extends from the drum section to the first end section, the second storage area to wind the section of the cable which extends from the drum section to the second end section. In this case, the storage areas are mutually offset, i.e. as a result are arranged one above the other, viewed in the direction of the drum axis.

Preferably, the cable is also arranged in a protective sheath. In this case, the protective sheath is wound or unwound together with the cable. The cable therefore always remains in the protective sheath.

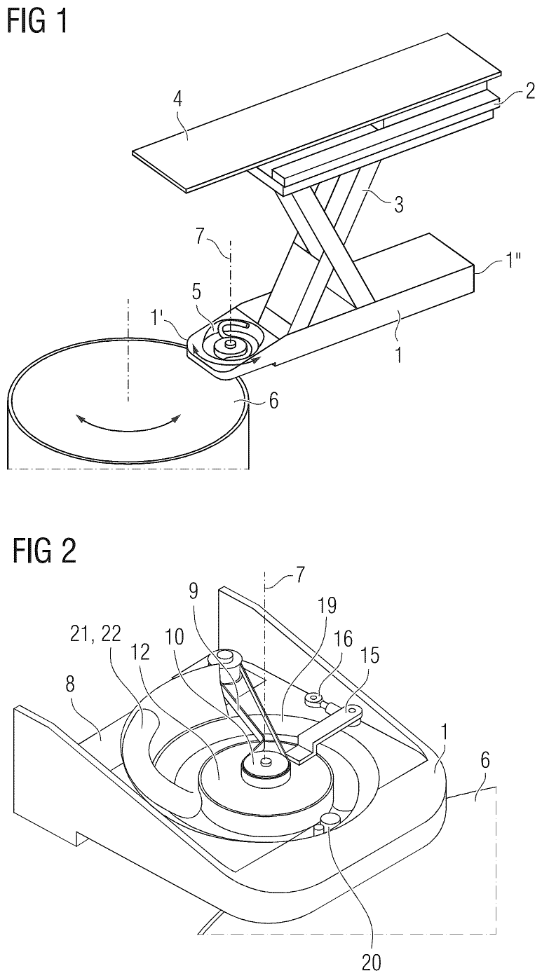

According to FIG. 1, a patient table for RT applications has a lower structure 1 and an upper structure 2. The lower structure 1 and the upper structure 2 are connected together via a lifting mechanism 3. The lifting mechanism 3 can be embodied, for example, as a scissor lift. However, other embodiments are also possible, for example as a hydraulic or pneumatic cylinder or as a screw which can be screwed into a threaded hole and which can be unscrewed by a motor. The lifting mechanism 3 is arranged below the upper structure 2.

A table top 4 is arranged on the upper structure 2. The table top 4 is dimensioned in such a way that an adult person (not represented) can be supported on it. The table top 4 therefore has a length of approx. 2 m or slightly more and a width of approx. 60 cm. The table top 4 can be arranged on the upper structure 2 in such a way that it can move in its longitudinal direction and potentially also in its transverse direction.

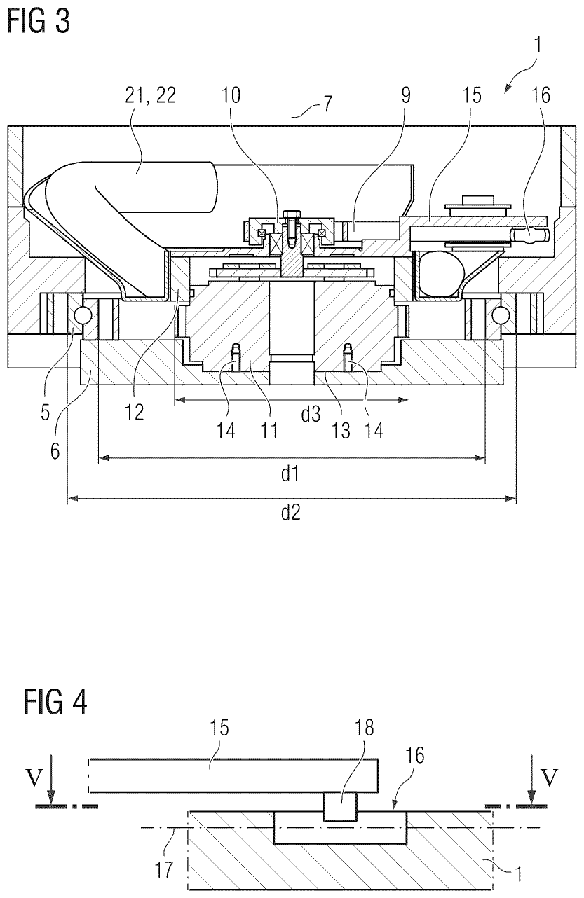

The lower structure 1 extends from one end 1′ to another end 1″. The lifting mechanism 3 is arranged between the one end and the other end 1′, 1″ of the lower structure 1. At the one end 1′, the lower structure 1—see also FIGS. 2 and 3—is supported on a foundation 6 via a pivot bearing 5. The lower structure 1 can consequently rotate about a vertical axis of rotation 7. The other end 1″ is not supported. In the present case, the foundation 6 can itself rotate about a further vertical axis of rotation.

In the present case, the rotatability of the foundation 6 is of secondary importance. The further vertical axis of rotation is therefore drawn in broken lines but is not provided with a reference numeral. Furthermore, this further vertical axis of rotation will not be discussed in more detail below either. Where an axis of rotation is mentioned below, it is in fact always the vertical axis of rotation 7 that is intended, and this is defined by the pivot bearing 5.

Where the terms “axial”, “radial” and “tangential” are used below, as a rule, they also always refer to the vertical axis of rotation 7. “Axial” is a direction parallel to the vertical axis of rotation 7. “Radial” is a direction orthogonal to the axial direction directly towards to the vertical axis of rotation 7 or away from it. “Tangential” is a direction which runs both orthogonal to the axial direction as well as orthogonally to the radial direction. “Tangential” therefore denotes a direction which, with a constant axial position and in a constant radial spacing from the vertical axis of rotation 7, is directed circularly around the vertical axis of rotation 7. If the terms “axial”, “radial” and “tangential” should, as an exception, not be based on the vertical axis of rotation 7, the axis to which the terms refer will be explicitly indicated in each case. In respect of this, then explicitly indicated axis, the terms “axial”, “radial” and “tangential” are defined in the same way.

The pivot bearing 5 has to be designed in such a way that it can receive and absorb the tilting moment, which [ ] from the lower structure 1 and the elements borne by the lower structure 1 (that is to say, as a result the lifting mechanism 3, the upper structure 2, the table top 4 and the person supported on the table top 4 and possibly further components arranged on the table top 4). The pivot bearing 5 therefore has appropriately large dimensions. In particular, the pivot bearing 5 has a relatively large internal diameter d1 and an even larger external diameter d2.

A drive 8 is arranged on the lower structure 1 to bring about a rotational movement of the lower structure 1 about the axis of rotation 7. The drive 8 acts—for example via a drive belt 9—on a drive shaft 10 of a gear 11. The drive shaft 10 acts on a driven shaft 12 of the gear 11. As a rule, the gear 11 brings about a clear reduction of, for example, 100:1 (that is to say, 100 revolutions of the drive shaft 10 bring about 1 revolution of the driven shaft 12). The reduction can also be even greater, for example can lie at 120:1 or even more, sometimes even at up to 200:1.

The gear 11 is a structural element different from the pivot bearing 5. The gear 11 has a (separate) gear casing 13. The gear casing 13 is non-rotatably arranged on the foundation 6—for example via screws 14. Based on the axis of rotation 6, the gear casing 13 is arranged radially inside the pivot bearing 5 and axially in the region of the pivot bearing 5. The gear casing 13 has a casing diameter d3. The drive shaft 10 and the driven shaft 12 are supported in the gear casing 13 of the gear 11. The casing diameter d3 is smaller than the internal diameter d1 of the pivot bearing 5.

An arm 15 is non-rotatably arranged on the driven shaft 12 for rotating the lower structure 1 about the axis of rotation 7. The arm 15 is connected to the lower structure 1, so when the drive shaft 10 rotates (and therewith also the driven shaft 12), the arm 15 exerts a torque on the lower structure 1. The lower structure 1 is consequently rotated about the axis of rotation 5. If the gear 11—more precisely: the axis of the driven shaft 12—were arranged exactly coaxially with the axis of rotation 7, a direct, rigid connection of the arm 15 to the lower structure 1 would be possible without problems. In practice, by contrast, a coaxiality of this kind cannot be achieved. To solve this problem, the arm 15 acts on the lower structure 1 via a coupling element 16. The coupling element 16 is embodied in such a way that via the coupling element 16, the torque exerted on the arm 15 is transferred to the lower structure 1 (and consequently the lower structure 1 is rotated), but a radial movement of the arm 15 relative to the lower structure 1 is enabled. For example, the coupling element 16 can be embodied as a lever corresponding to the representation in FIG. 2, which lever substantially extends tangentially to the axis of rotation and has two ends. In this case, one each of the two ends of the lever is articulated to the lower structure 1 and the arm 15.

A further, alternative possibility to the embodiment of the coupling element 16 will be explained below in connection with FIGS. 4 and 5. According to FIGS. 4 and 5, the coupling element 16 is embodied as a slot with a radially-running longitudinal axis 17. A pin 18 of the arm 15 is led into the slot. The pin 18 extends in the axial direction. The pin 18 is matched to the width of the slot, so it can be led into the slot in a precisely-fitting manner. The pin 18 is therefore arranged in the slot free from play in the transverse direction of the slot (corresponds to the current tangential direction respectively). By contrast, the pin 18 can move in the slot in the longitudinal direction of the slot (corresponds to the current radial direction respectively). Analogously, the inverse embodiment is also possible in which the slot is an integral part of the arm 15 and the pin 18 an integral part of the coupling element 16.

The coupling element 16 can also be embodied as a tensile/pressure force rigid element which is fastened with non-positive or positive fit tangentially to the end of the arm 15 and also to the lower structure 1, for example via screwing. The coupling element 16 can be embodied, for example, as a sheet metal strip which in the radial direction of the absorption of the force of the torque acts as a rigid transfer element, but can in the process compensate small radial tolerances owing to concentricity inaccuracies. By way of a non-positive fastening which is free from play, a coupling element 16 of this kind can transfer the torque from the gear 11 to the lower structure 1 free from play and rigidly, simultaneously compensate small inaccuracies in the concentricity and is additionally a very inexpensive element whose rigidity can be designed over the sheet metal thickness, such as the width or shape, for the respective application.

Some advantageous embodiments of the present invention can be found in FIG. 1 to 3.

Thus, owing to the fact that the casing diameter d3 is smaller than the internal diameter d1 of the pivot bearing 5, an annular gap 19 is formed between the pivot bearing 5 and the gear casing 13. A feedthrough 20 is arranged in the annular gap 19. A cable 21 is guided from the foundation 6 into the lower structure 1 via the feedthrough 20. The drive 8 is supplied with electrical energy via the cable 21.

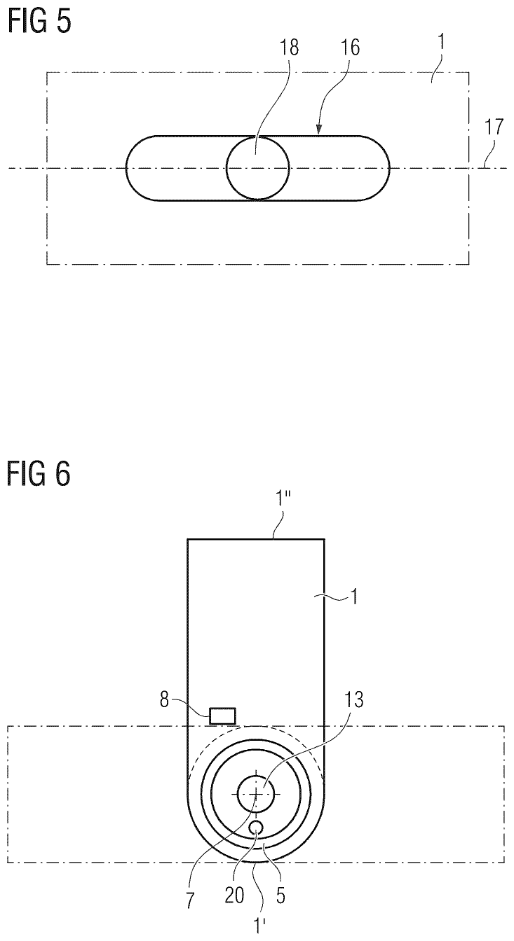

The lower structure 1 can be rotated between two end positions via the drive 8. FIG. 6 shows the two end positions in broken lines. The two end positions are usually exactly or at least approximately diametrically opposed in respect of the axis of rotation 7. Furthermore, a middle rotational position between the two end positions is marked in FIG. 6 by solid lines. The middle rotational position is preferably equidistant or at least approximately equidistant (based on the angle of rotation) from the two end positions. If the lower structure 1 is in the middle rotational position, the gear casing 13 is arranged between the other end 1″ of the lower structure 1 and the feedthrough 20. Furthermore, according to FIG. 6 (see also FIG. 2), the drive 8 is arranged between the other end 1′ of the lower structure 1 and the pivot bearing 5.

In the previously explained embodiment, the cable 21 is embodied as a cable carrier—this is particularly clear in FIGS. 2 and 3. The cable carrier is placed in the annular gap 19 or removed from the annular gap 19 depending on the direction in which the lower structure 1 is rotated via the drive 8. Preferably, the cable 21 is also arranged in a protective sheath 22. In this case, the protective sheath 22, together with the cable 21, is placed in the annular gap 19 or removed from the annular gap 19. The protective sheath 22 can be embodied, for example, as what is known as a flexible hose or what is known as triflex chain. In the case of the protective sheath 22, the cable 21 itself is loose in the protective sheath 22.

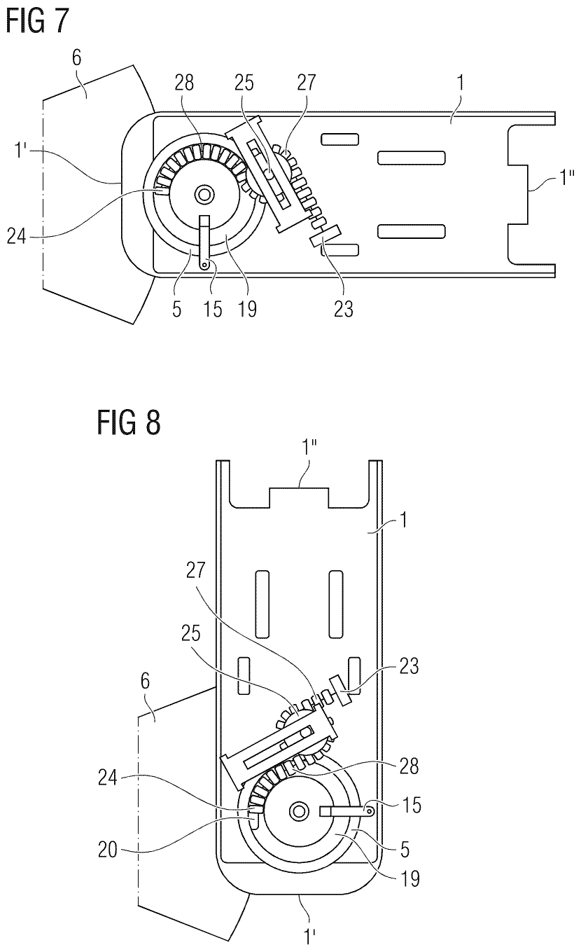

An alternative embodiment of the guidance of the cable 21 and associated advantageous embodiments will be explained below in connection with FIG. 7 to 10.

According to FIG. 7, a first end section 23 of the cable 21 is securely arranged on the lower structure 1. Analogously, a second end section 24 of the cable 21 is arranged in the region of the feedthrough 20. Furthermore, a cable drum 25 is supported on the lower structure 1. Supporting of the cable drum 25 is such that firstly the cable drum 25 can move translationally and secondly, can rotate about a drum axis 26 (see FIG. 10). The specific translational path of the cable drum 25 can be linear or curved. As a rule, it is brought about by an appropriate slotted guide. The orientation of the drum axis 26 can be as needed. Often the drum axis 26 is vertically oriented.

A particular section of the cable 21—hereinafter referred to as a drum section—is fixed inside the cable drum 25. The drum section is not represented in the figure. However, the drum section is located between the first and the second end sections 23, 24. Starting from the drum section, a first intermediate section 27 extends to the first end section 23. Analogously, starting from the drum section, a second intermediate section 28 extends to the second end section 24. Viewed from the first end section 23 to the second end section 24, are therefore situated, in each case adjoining one another and the end sections 23, 24, first of all the first intermediate section 27, then the drum section and finally the second intermediate section 28.

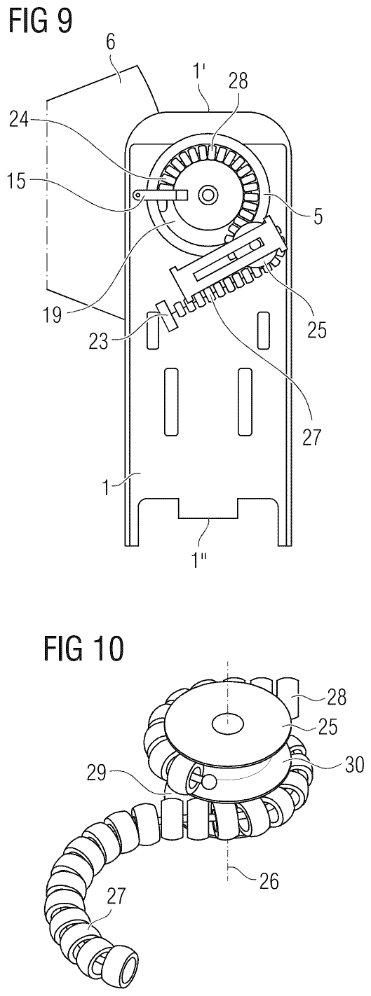

The cable drum 25 is wound with the two intermediate sections 27, 28 in such a way that when the cable drum 25 rotates about the drum axis 26, the cable drum 25, depending on the direction of rotation of the cable drum 25, either unwinds or winds the cable 21 in the direction of the first as well as the second end section 23, 24. FIG. 8 shows the lower structure 1 in the one end position in which the cable 21 is wound to the maximum on the cable drum 25. Conversely, FIG. 9 shows the lower structure 1 in the other end position in which the cable 21 is unwound to the maximum from the cable drum 25. The cable drum 25 is preferably spring-loaded in such a way that it tensions the intermediate section 27, 28. Alternatively or in addition, a forced rotation of the cable drum 25 can be implemented. One example of an implementation of this kind is an embodiment of the slotted guide mentioned above as a pinion in connection with a toothed wheel arranged on the cable drum 25, which wheel meshes with the pinion and brings about the rotation of the cable drum 25.

Independently of the direction of rotation of the cable drum 25, either both intermediate sections 27, 28 are wound onto the cable drum 25 or else both intermediate sections 27, 28 are unwound from the cable drum 25 therefore. By contrast, the case where one of the two intermediate sections 27, 28 is wound onto the cable drum 25 and the other of the two intermediate sections 27, 28 is unwound from the cable drum 25 cannot occur. This is evident particularly clearly in FIG. 10 in which solely the cable drum 25 and the intermediate sections 27, 28 are represented. The curved profile of the first intermediate section 27 can be achieved, for example, by a corresponding guide contour. The guide contour is not represented in FIG. 10.

A plurality of advantageous embodiments of the cable 21 and the cable drum 25 is also particularly clearly evident in FIG. 10.

To explain the first advantageous embodiment, first of all a first and a second contact region of the cable drum 25 will be defined below. The two contact regions can be static or change depending on the rotational position of the lower structure 1. The first contact region is the location of the cable drum 25 at which, in the direction of the first end section 23, a first section of the cable 21 (or the first intermediate section 27), wound onto the cable drum 25, merges into a section of the cable 21 (or the first intermediate section 27) which extends in the direction of the first end section 23. Analogously, the second contact region is the location of the cable drum 25 at which, in the direction of the second end section 24, a second section of the cable 21 (or the second intermediate section 28), wound onto the cable drum 25, merges into a section of the cable 21 (or the second intermediate section 28) which extends in the direction of the second end section 24. It is evident that the first contact region and the second contact region are exactly or at least approximately diametrically opposed in respect of the drum axis 26.

Furthermore, the cable drum 25 has a first storage area 29 and a second storage area 30. The first storage area 29 serves to wind the first intermediate section 27. Analogously, the second storage area 30 serves to wind the second intermediate section 28. It is evident that, viewed in the direction of the drum axis 26, the storage areas 29, 30 are mutually axially offset.

Finally, the cable 21—similar to in the embodiment in which the cable 21 is placed in the annular gap 19—is arranged in a protective sheath 22. In the case of the embodiment of FIG. 7 to 10, the protective sheath 22, together with the cable 21, is wound onto the cable drum 25 or is unwound from it. The cable 21 itself is again loose in the protective sheath 22 in this case too. The tensile stress occurs only in the protective sheath 22.

Example embodiments many advantages. Thus, for example, elements can be used for the pivot bearing 5 and the gear 11, which are optimally dimensioned for the respective task. This results in a cost optimization. Furthermore, standardized components can be used. The installation space for the pivot bearing 5 and the gear 11 can be used optimally and flexibly. Advantages result in respect of the costs, assembly, adjustment and also testing during initial operation owing to the low requirements placed on the precision of the assembly interfaces. The flexibility of operation of the RT gantry increases owing to the low installation space that is required, in particular the low installation height. The pivot bearing 5 and the gear 11 can be assembled and also be replaced independently of each other. Owing to the independent assembly, for example the lower structure 1 can also firstly rotate in the pivot bearing 5 manually, i.e. independently of the drive 8 and the gear 11, during the course of production of the patient table. The pivot bearing 5 can firstly be tested thereby. Analogously, the gear 11 can also be tested separately. The required quantity of cable 21 can be easily and compactly stored both in the embodiment of the cable 21 as a cable carrier as well as when the cable drum 25 is used. In particular when the cable drum 25 is used, the installation height is still kept low in the case of a vertical orientation of the drum axis 26 because the minimum bending radius of the cable 21 no longer influences the installation height.

Although the invention has been illustrated and described in detail by the preferred exemplary embodiments, it is not limited by the disclosed examples and a person skilled in the art can derive other variations herefrom without departing from the scope of the invention.

Independent of the grammatical term usage, individuals with male, female or other gender identities are included within the term.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,“ ”connected,” “engaged,” “interfaced,” and “coupled. ” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Specific structural and functional details disclosed herein are merely representative for purposes of describing example embodiments. The present invention may, however, be embodied in many alternate forms and should not be construed as limited to only the embodiments set forth herein.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A patient table comprising:
a lower structure;
an upper structure connected to the lower structure via a lifting mechanism;
a table top on the upper structure, on which an adult person can be supported, wherein
a first end of the lower structure is supported on a foundation via a pivot bearing, such the lower structure is rotatable about a vertical axis of rotation,
another end of the lower structure is not supported,
the lifting mechanism is between the one and the other end of the lower structure and below the upper structure;
a drive on the lower structure, the drive configured to act on a drive shaft of a gear, wherein
a rotation of the drive shaft brings about a rotation of a driven shaft,
the gear includes a gear casing which is non-rotatably arranged on the foundation and in which the drive shaft and the driven shaft are supported; and
an arm is non-rotatably arranged on the driven shaft and is connected to the lower structure, so when the drive shaft rotates, the arm exerts a torque on the lower structure, so the lower structure is rotated about the axis of rotation, wherein
the gear is a structural element different from the pivot bearing,
based on the axis of rotation, the gear casing is arranged radially inside the pivot bearing and axially in a region of the pivot bearing, and
the arm is configured to act on the lower structure via a coupling element, via which the torque exerted on the arm is transferred to the lower structure while a radial movement of the arm relative to the lower structure is still enabled.

2. The patient table of claim 1, wherein
the coupling element is a slot with a radially-running longitudinal axis and a pin of the arm is led into the slot, or
the coupling element is a pin which is led into a slot, introduced into the arm, the slot having a radially-running longitudinal axis.

3. The patient table of claim 1, wherein the coupling element is a lever with two ends and one each of the two ends of the lever is articulated to the lower structure and the arm, the lever extending substantially tangentially to the axis of rotation.

4. The patient table of claim 1, wherein the pivot bearing has an internal diameter and the gear casing has a casing diameter and the casing diameter of the gear casing is smaller than the internal diameter of the pivot bearing such that an annular gap is formed between the pivot bearing and the gear casing, and that a feedthrough is in the annular gap, via which a cable is guided from the foundation into the lower structure, via which cable the drive is supplied with electrical energy.

5. The patient table of claim 4, wherein
the lower structure is rotatable between two end positions via the drive, and
when the lower structure is in a central rotational position between the two end positions, the gear casing is between the other end of the lower structure and the feedthrough.

6. The patient table of claim 4, wherein the drive is between the other end of the lower structure and the pivot bearing.

7. The patient table of claim 4, wherein the cable is a cable carrier which, depending on a direction in which the lower structure is rotated via the drive, is placed in the annular gap or is removed from the annular gap.

8. The patient table of claim 7, wherein the cable is in a protective sheath and the protective sheath, together with the cable, is in the annular gap or is removed from the annular gap.

9. The patient table of claim 4, wherein
a first end section of the cable is secured on the lower structure and a second end section of the cable is in the region of the feedthrough,
a cable drum is on the lower structure such that the cable drum is moveable translationally and rotatable about a drum axis,
a drum section of the cable, which is between the first and the second end sections, is fixed inside the cable drum and when the cable drum rotates about the drum axis, the cable drum, depending on a direction of rotation of the cable drum, unwinds or winds the cable in the direction of the first section and the second end section.

10. The patient table of claim 9, wherein a first contact region of the cable drum at which, in the direction of the first end section, a first section of the cable wound onto the cable drum merges into a section of the cable which extends in the direction of the first end section, and a second contact region of the cable drum at which, in the direction of the second end section, a second section of the cable wound onto the cable drum merges into a section of the cable which extends in the direction of the second end section, are located approximately diametrically opposed with respect to the drum axis.

11. The patient table of claim 9, wherein the cable drum includes,
a first storage area for winding the section of cable which extends from the drum section to the first end section, and
a second storage area for winding the section of cable which extends from the drum section to the second end section and the storage areas are mutually offset, in the direction of the drum axis.

12. The patient table of claim 9, wherein the cable is in a protective sheath and the protective sheath, with the cable, is wound or unwound.

13. The patient table of claim 3, wherein the pivot bearing has an internal diameter and the gear casing has a casing diameter and the casing diameter of the gear casing is smaller than the internal diameter of the pivot bearing such that an annular gap is formed between the pivot bearing and the gear casing, and that a feedthrough is in the annular gap, via which a cable is guided from the foundation into the lower structure, via which cable the drive is supplied with electrical energy.

14. The patient table of claim 13, wherein
the lower structure is rotatable between two end positions via the drive, and
when the lower structure is in a central rotational position between the two end positions, the gear casing is between the other end of the lower structure and the feedthrough.

15. The patient table of claim 14, wherein the drive is between the other end of the lower structure and the pivot bearing.

16. The patient table of claim 15, wherein the cable is a cable carrier which, depending on a direction in which the lower structure is rotated via the drive, is placed in the annular gap or is removed from the annular gap.

17. The patient table of claim 16, wherein the cable is in a protective sheath and the protective sheath, together with the cable, is in the annular gap or is removed from the annular gap.
